(19) United States
(12) Patent Application Publication
(10) Pub. No.: US 2012/0088265 A1
(43) Pub. Date: Apr. 12, 2012

(54) Mold Pathogen Detection Apparatus

(76) Inventors: Rajesh Guntupalli, Auburn, AL (US); Nilmini Viswaprakash, Auburn, AL (US)

(21) Appl. No.: 12/899,910

(22) Filed: Oct. 7, 2010

(51) Int. Cl. C12Q 1/04; C12M 1/34

(52) U.S. Cl. 435/34; 435/288.7

(57) ABSTRACT

The present invention is a mold pathogen detection apparatus in which an agar strip is exposed to the environment being tested for a period of time and then exposed to a non-UV light to determine the amount of light passing through the agar strip.

### FIELD OF INVENTION

The present invention relates to the field of detectors for adverse conditions, and more specifically to a mold pathogen detection apparatus.

### BRIEF DESCRIPTION OF THE DRAWINGS

FIG. 1 is an exemplary embodiment of a single-test mold pathogen detection apparatus.

FIG. 2 is an exemplary embodiment of a multiple test mold pathogen detection apparatus using agar plates.

FIG. 3 is an exemplary embodiment of a multiple test mold pathogen detection apparatus using agar strips.

FIG. 4 is an exemplary embodiment of a multiple test mold pathogen detection apparatus with a UV light.

FIG. 5 is an exemplary embodiment of an enclosed multiple test mold pathogen detection apparatus.

### GLOSSARY

As used herein, the terms “agar plate” or “agar strip” refer to an agar-based growth medium contained on a plate or strip and used to culture mold colonies. Agar plates and agar strips may include agar compositions including, but not limited to, potato dextrose agar, sabouraud agar, hay infusion agar, malt extract agar and combinations thereof. Agar plates and agar strips may also contain antibiotics for specific molds or classes of molds or other selective compounds to allow for growth of a desired test mold. Agar plates and agar strips may also contain other compounds to prohibit the growth of unwanted bacteria.

As used herein, the term “mold” means multicellular fungi that grow in genetically identical colonies.

As used herein, the term “mold spore” means a reproductive structure of a mold.

As used herein, the term “photodetector” means any device in the art capable of detecting and measuring light intensity.

### BACKGROUND

Molds are multicellular fungi that grow in genetically identical colonies. Molds can be found both indoors and outdoors, and grow best in warm, humid conditions, although mold spores can survive harsh conditions that usually do not support mold growth.

Airborne mold and mold spores are known to cause health problems, particularly in people with sensitivities towards molds. Exposure to molds and mold spores may cause simple allergic responses, such as sneezing, itchy eyes and stuffiness, but may also cause severe problems including fever, wheezing, and upper respiratory tract symptoms. Prolonged exposure to molds and mold spores may also cause chronic lung illnesses, such as mold infections and asthma. People may also develop rashes or other skin irritations when exposed to molds and mold spores.

Because molds are found both indoors and outdoors, at all times of the year, it is sometimes impossible for people to completely avoid exposing themselves to mold and mold spores. However, there are things people can do to limit mold exposure. For example, running dehumidifiers in the home may prevent mold from growing by making conditions unfavorable for growth. Applying mold inhibitors to paints and other wall treatments and decreasing the amount of carpeted floor space also help to limit favorable mold-growing conditions. When it comes to exposure to outdoor molds, the best measure to take is avoiding damp, mold-producing areas, although outdoor mold growth can be somewhat controlled by removing all decomposing matter from the area (such as compost piles) and keeping firewood piles dry.

Despite all precautions, molds sometimes grow in unseen or hard to control areas. Even after molds have been removed, mold spores may still survive and settle on surfaces not suited for mold growth. People with sensitivities to molds may therefore still show allergy-like symptoms even though there is no sign of mold growth. As a result, many people may be treated for allergies year-round when the actual culprit, mold, is still present, but unseen.

Even though people can develop serious health problems from mold exposure, there are currently no official standards or recommendations for airborne mold limit or threshold concentrations. To determine whether an indoor location has a significant air quality problem due to airborne mold, the concentration of airborne mold found indoors is compared to that found outdoors nearby. If the concentration of mold indoors is significantly greater than the concentration of mold outdoors, there is likely an indoor mold problem.

Specific industries may, however, set their own internal acceptable mold concentration limits. Hospitals, for example, may set tolerable limits for specific molds that are known to interfere with patient recovery. Similarly, food and pharmaceutical plants may monitor airborne mold concentrations for quality control reasons. Many times food and pharmaceutical plants begin monitoring mold concentrations in response to consumer complaints.

Individuals with severe mold intolerances or weakened immune systems may also wish to monitor indoor airborne mold concentrations and maintain minimal levels of mold in living spaces and work environments. Parents of susceptible children, in particular, may wish to monitor indoor mold concentrations and limit the child's exposure.

It is desirable to develop an apparatus capable of detecting molds and mold spores over a period of time.

It is further desirable to develop an apparatus capable of detecting molds and mold spores over a period of time that alerts users when airborne mold concentrations surpass a certain threshold.

### SUMMARY OF THE INVENTION

The present invention is a mold pathogen detection apparatus in which an agar strip is exposed to the environment being tested for a period of time and then exposed to a non-UV light to determine the amount of light passing through the agar strip.

### DETAILED DESCRIPTION OF INVENTION

For the purpose of promoting an understanding of the present invention, references are made in the text to exemplary embodiments of mold pathogen detection apparatus, only some of which are described herein. It should be understood that no limitations on the scope of the invention are intended by describing these exemplary embodiments. One of ordinary skill in the art will readily appreciate that alternate but functionally equivalent materials and methods may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

It should be understood that the drawings are not necessarily to scale; instead emphasis has been placed upon illustrating the principles of the invention. In addition, in the embodiments depicted herein, like reference numerals in the various drawings refer to identical or near identical structural elements.

Moreover, the terms “substantially” or “approximately” as used herein may be applied to modify any quantitative representation that could permissibly vary without resulting in a change in the basic function to which it is related.

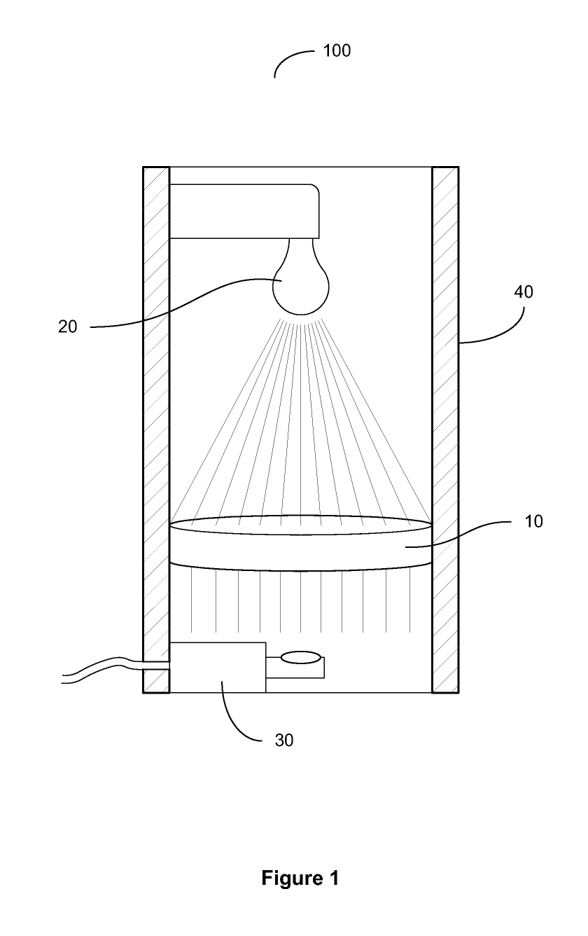

FIG. 1 illustrates an exemplary embodiment of mold recognition element 100 with a single agar plate 10. Agar plate 10 is placed in chamber 40 between non-UV light source 20 and photodetector 30 to obtain a base reading of the amount of the light intensity that passes through agar plate 10. Agar plate 10 is then exposed to an environment desired to be monitored for mold and mold spores and allowed to rest for a period of time. To determine mold growth, agar plate 10 is put back in chamber 40 between non-UV light source 20 and photodetector 30, and a light intensity reading is taken. If the second light intensity reading is the same as the first, there is no mold growth on agar plate 10. If the second light intensity reading indicates less light is passing through agar plate 10, mold growth is present. Agar plate 10 may be monitored over a period of time to determine the rate of mold growth.

In the exemplary embodiment shown, agar plate 10 is made from potato dextrose agar. In other embodiments, agar plate 10 may be any composition known in the art to foster the growth of molds, including, but not limited to, sabouraud agar, hay infusion agar and malt extract agar. In still other embodiments, agar plate 10 may be adapted to grow only a specific mold or group of molds by including antibiotics in the agar composition. The pH and other characteristics of the agar composition may also be varied to provide growth environments specific to individual molds or a group of molds.

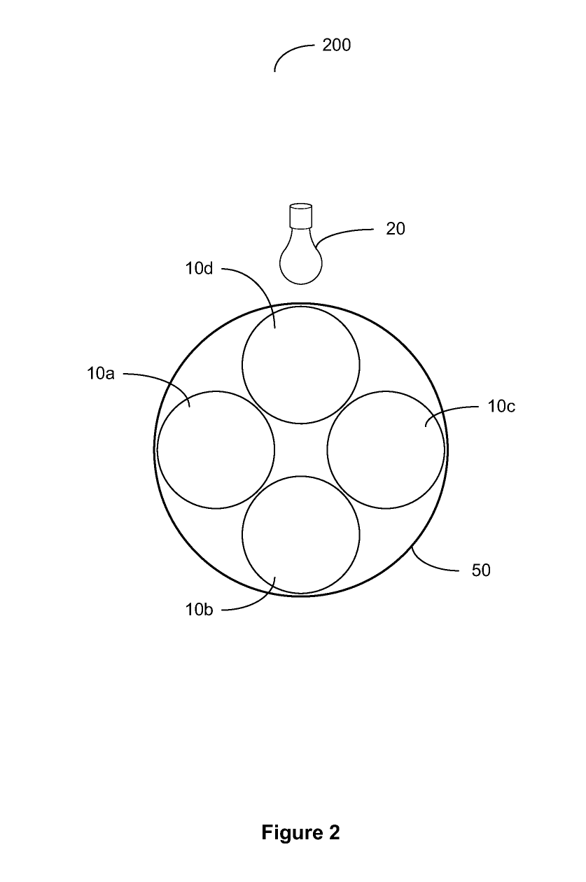

Mold recognition elements may also include more than one agar plate, as illustrated in the exemplary multi-plate mold recognition element 200 in FIG. 2. In the exemplary embodiment shown in FIG. 2, four agar plates 10a, 10b, 10c, and 10d rotate on rotating disc 50 to first be exposed to the environment and then slide under non-UV light source 20. Photodetector 30 (not shown) detects the amount of light passing through agar plates 10a-10d to detect mold growth. To prevent outside light from reaching photodetector 30 (not shown), non-UV light source 20 and photodetector 30 (not shown) are shielded within chamber 40, which is not shown in order to better illustrate multiple test mold recognition element 200.

Rotating disc 50 may be manually turned to move agar plates or mechanically turned by any means known in the art to turn rotating disc 50. In other still other embodiments, rotating disc 50 may also include a timing mechanism adapted to move rotating disc 50 at predetermined intervals.

In the embodiment shown in FIG. 2, agar plates 10a, 10b and 10c are each exposed to the environment, while agar plate 10d is in position to be exposed to non-UV light source 20. In further exemplary embodiments, only one agar plate is exposed to the environment at a time, while the other non-used agar plates remain hygienically sealed or covered until ready for exposure.

In the embodiments shown in FIGS. 1 and 2, agar plates are shown to be standard sized Petri dishes. In still other embodiments, agar plates may be of any size, including agar strips that can be as small as 1-2 cm in diameter. In still further exemplary embodiments, agar strips may be rectangular in shape and as small as 2 cm by 2 cm.

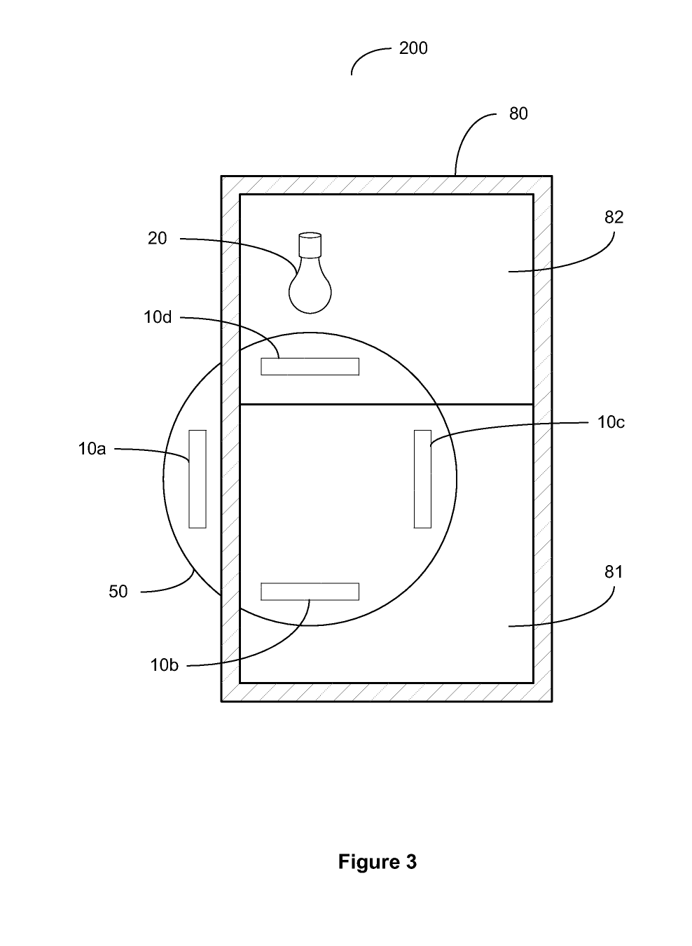

FIG. 3 is an exemplary embodiment of multiple test mold recognition element 200 using agar strips 10a-10d instead of plates. Agar strips are cheaper and smaller than agar plates, allowing for lower manufacturing costs. Agar strips 10a-10d rotate on rotating disc 50 and are exposed to the environment one at a time. Agar strip 10a is exposed to the environment, while agar strips 10b and 10c remain covered or sealed until rotated into the position currently occupied by 10a. Agar strip 10d is position between non-UV light source 20 and photodetector 30 (not shown).

In the exemplary embodiment shown in FIG. 3, multiple test mold recognition element 200 is shown in casing 80. Casing 80 contains chambers 81 and 82. Agar strips 10b and 10c are shown within chamber 81 of casing 80, which shelters unused agar strips until they are to be exposed to the environment. Chamber 82 of casing 80 houses non-UV light source 20 and photodetector 30 (not shown) to prevent external light from reaching photodetector 30. Agar strip 10a is not contained within casing 80 because it is exposed to the environment.

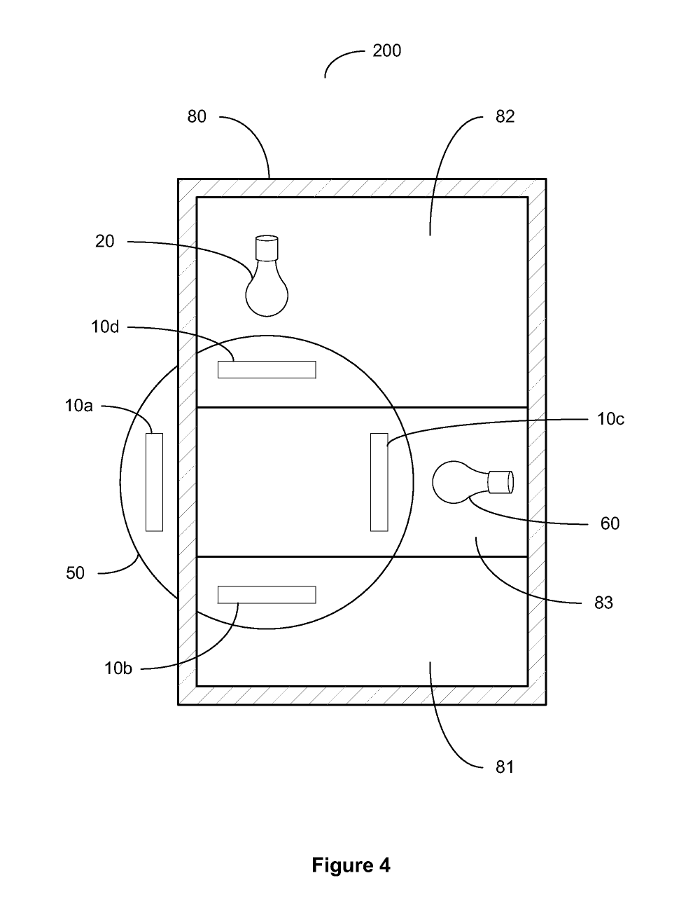

FIG. 4 is an exemplary embodiment of multiple test mold recognition element 200 using agar strips 10a-10d and UV light source 60. Agar strips 10a-10d rotate on rotating disc 50 and are exposed to the environment one at a time. In the exemplary embodiment shown, agar strip 10a is exposed to the environment, while agar strip 10b-10d are shown within casing 80. Casing 80 is shown with three chambers 81, 82 and 83. Chamber 81 shelters agar strip 10b, which is not yet exposed to the environment. Agar strip 10d is shown between non-UV light source 20 and photodetector 30 (not shown) to determine the amount of light passing through agar strip 10d. After photodetector 30 completes a reading, rotating disc 50 rotates agar strip to the position occupied by agar strip 10c in the exemplary embodiment shown. Agar strip 10c is exposed to UV light from UV light source 60, which kills any mold growth. Killing mold growth prevents reintroducing mold to the environment.

In the exemplary embodiments shown in FIGS. 3 and 4, agar strips 10a-10d may be replaced after testing. Casing 80 may have an access compartment or any other device or structure known in the art to allow access to agar strips 10a-10d to allow replacement. In further embodiments, rotating disc 50 may be replaced with one containing new agar strips. Casing 80 may also have access points to replace light sources.

In the exemplary embodiments shown in FIGS. 2, 3 and 4, multiple test mold recognition element 200 is shown with four agar plates or strips. In further embodiments, more or fewer agar plates or strips may be used. Similarly, casing 80 may be adapted to expose more than one agar strip and/or allow an exposed agar strip to incubate inside a separate chamber of casing 80 to avoid contaminating the other agar strips. In still other embodiments, multiple test mold recognition element 200 may further include a heating element. While mold grows at room temperature, the growth rate increases in warmer, humid climates. A heating element which may enhance the speed of mold detection may be adapted to turn on and off depending on how fast testing and detection needs to be complete.

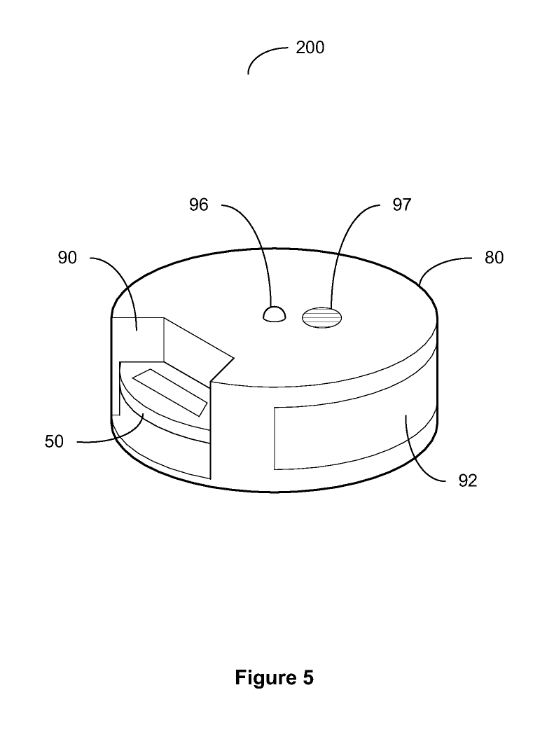

FIG. 5 illustrates an exemplary embodiment of multiple test mold recognition element 200 contained in casing 80. In the exemplary embodiment shown, casing 80 contains exposure point 90 which is open to the environment to allow agar strips to be exposed to the environment. Also shown is agar strip access point 92, which opens to reveal rotating disc 50 so agar strips may be replaced. In the exemplary embodiment shown, agar strip access point 92 is adapted to function similar to a DVD or CD player tray. In still other exemplary embodiments, agar strip access point 92 may be a removable cover, slide opening or any other structure known in the art to provide access to agar strips.

In the exemplary embodiments shown in FIGS. 1-5, each agar plate or agar strip may have an incubation period of up to 8 hours or more after exposure the environment and before being exposed to the non-UV light source for mold growth detection. Similarly, each agar plate or agar strip may be exposed to the non-UV light multiple times for multiple light intensity readings. Agar plates and agar strips may be monitored periodically for mold growth for up to 5 days. Monitoring mold growth over a period of days helps establish the initial concentration of mold the agar plate or strip was exposed to.

In the exemplary embodiment shown in FIG. 5, casing 80 is designed to look like a standard smoke detector, including an audible alarm 97 and light 96 which will sound and blink, respectively, if the light detected by a photodetector falls below a certain amount to indicate mold growth above a given concentration. In further embodiments, casing 80 may be adapted to look like other household objects, such as nightlights or air freshener devices. In still further embodiments, casing 80 may be adapted to be mounted in air conditioning units, floor cleaners (such as vacuum cleaning devices) or other devices known to potentially spread molds. In still further embodiments, mold recognition elements and multiple test mold recognition elements may be adapted and incorporated into home security systems for online monitoring.

What is claimed is:

1. A mold pathogen detection apparatus comprised of:
at least one non-UV light source;
at least one photodetector; and
at least one agar strip.

2. The apparatus of claim 1 which further includes a UV light source.

3. The apparatus of claim 1 which further includes an audible alarm.

4. The apparatus of claim 1 which further includes a heating element.

5. The apparatus of claim 1 wherein said at least one agar strip is further adapted to include antibiotics.

6. The apparatus of claim 1 wherein said at least one agar strip is comprised of an agar composition from the group consisting of potato dextrose agar, sabouraud agar, hay infusion agar, malt extract agar and combinations thereof.

7. The apparatus of claim 1 wherein said at least one agar strip is disposable.

8. A mold pathogen detection apparatus comprised of:
a housing with an exposure aperture;
at least one non-UV light source within said housing;
at least one photodetector within said housing;
at least one agar strip; and
a rotating disc.

9. The apparatus of claim 8 wherein said housing is adapted to mount to a surface selected from a group consisting of a wall, a ceiling, a door, a window, a piece of furniture, an air vent, a duct line, a floor cleaning device, a cold air return, an air conditioner unit, and a fan.

10. The apparatus of claim 8 wherein said rotating disc is adapted to removeably secure said at least one agar strip and rotate said at least one agar strip from said exposure aperture past said non-UV light source.

11. The apparatus of claim 8 which further includes a UV light source within said housing.

12. The apparatus of claim 8 which further includes a heating element within said housing.

13. The apparatus of claim 8 wherein said at least one agar strip is further adapted to include antibiotics.

14. The apparatus of claim 8 wherein said at least one agar strip is comprised of an agar composition from the group consisting of potato dextrose agar, sabouraud agar, hay infusion agar, malt extract agar and combinations thereof.

15. The apparatus of claim 8 which further includes at least one alerting device selected from the group consisting of an audible alarm, a blinking light and a solid light.

16. The apparatus of claim 8 which further includes a timing mechanism adapted to control said rotating disc.

17. The apparatus of claim 8 wherein said at least one agar strip is disposable.

18. A method for detecting mold comprising the steps of:
exposing an agar strip to a non-UV light source;
detecting the base amount of light passing through said agar strip;
exposing said agar strip to an environment;
exposing said agar strip to said non-UV light source;
detecting the amount of light passing through said agar strip;
comparing said amount of light passing through said agar strip to said base amount of light passing through said agar strip.

19. The method of claim 18 which further includes the step of heating said agar strip.

20. The method of claim 18 which further includes the step of exposing said agar strip to a UV light source.
